(19) United States
(12) Patent Application Publication
(10) Pub. No.: US 2026/0144656 A1
(43) Pub. Date: May 28, 2026

(54) METHODS AND SYSTEMS FOR AN INTRACRANIAL STENT

(71) Applicant: Ceroflo Limited, Galway (IE)

(72) Inventors: Brendan CASEY, Galway (IE); Eamon Oliver BRADY, County Galway (IE); Sven Tommy ANDERSSON, Älvsjö Älvsjö (SE)

(73) Assignee: Ceroflo Limited, Galway (IE)

(21) Appl. No.: 19/400,610

(22) Filed: Nov. 25, 2025

Related U.S. Application Data
(60) Provisional application No. 63/725,628, filed on Nov. 27, 2024.

(51) Int. Cl. A61F 2/89

(52) U.S. Cl. CPC A61F 2/89; A61F 2230/0019; A61F 2230/0023; A61F 2250/0036; A61F 2250/0039; A61F 2250/0098

(57) ABSTRACT

An intracranial stent may include one or more rings of struts at a proximal end portion, a distal end portion, a central portion, a proximal transition portion between the proximal end portion and central portion, and a distal transition portion between the distal end portion and central portion. The one or more rings at the central portion may have a diameter that is smaller than a diameter of the one or more rings of struts at the proximal and/or distal end portion. An intracranial stent may include a first ring of connectors connecting a first ring of the one or more rings of struts at the proximal end portion with a second ring of the one or more rings of struts at the proximal transition portion, each connector of the first ring of connectors may include a first curved section, a straight section, and a second curved section.

### CROSS-REFERENCE TO RELATED APPLICATIONS

This application claims priority to U.S. Provisional Application No. 63/725,628, filed Nov. 27, 2024, which is hereby incorporated by reference in its entirety.

### TECHNICAL FIELD

The present disclosure relates generally to systems and methods for treating an intracranial blood vessel, and more particularly, to methods and systems including an intracranial stent.

### BACKGROUND

Intracranial atherosclerotic disease (ICAD) refers to the presence of a stenosis that results in a narrowing of an artery, vein, or other blood vessel (hereinafter referred to collectively as “vessels”) within the brain. During early stages of atherosclerosis, fatty material collects along the walls of vessels. The fatty material thickens, forming plaque and resulting in narrowing of the vessel. In some cases, the stenosis obstructs the vessel, preventing blood flow through the vessel. This can lead to thromboembolic or hemodynamic ischemic stroke, a leading cause of disability worldwide.

Stents are used to treat atherosclerotic disease in various vessels of the body. Stents are formed in a variety of shapes and sizes for performing different functions once implanted. For example, some stents provide a scaffold with the goal of reducing the occlusion size to facilitate the restoration of flow and preventing the generation of emboli. Intracranial stents, in particular, are specifically designed for treatment of intracranial atherosclerotic disease. Stenting acts to widen the lumen of the stenosis thereby increasing blood flow through the vessel. Sub-maximal stents, stents designed to expand a vessel to a diameter that is less than its maximum healthy diameter, include a midsection having a diameter that is reduced when compared to the non-diseased vessel diameter. The midsection is positioned in the stenosis and deployed in the vessel. It is particularly important for submaximal stents to have accurate deployment positioning so that the stenosis is contained by the mid-section, for example within an annular space that surrounds the mid-section following deployment.

When deploying submaximal stents, the microcatheter can contact the submaximal stent and create undesired torque on the microcatheter. The design of the submaximal stent, including the shape of connectors between the rings, can impact the torque exerted on the microcatheter by the stent during deployment. The radial force of an intracranial stent is key to the ability of the stent to dilate the lumen of the stenosis The design of the stent, the stent wall thickness, the strut length, and the strut width, can affect the radial force profile of the submaximal stent. It is challenging to design a submaximal stent with a radial force profile along its length that has a low radial force applied to the walls of a vessel and a high radial force applied to the obstruction (e.g., plaque) in the vessel. The submaximal stent is designed to create low endothelial shear stress at the area of obstruction in the vessel.

The present disclosure may overcome one or more of these above-referenced challenges, or other challenges in the art. The scope of protection is, however defined by the claims, and not by a solution to a particular problem described herein or other problem in the art.

### SUMMARY OF THE DISCLOSURE

In some aspects, the techniques described herein relate to an intracranial stent, including: one or more rings of struts at a proximal end portion of the intracranial stent; one or more rings of struts at a distal end portion of the intracranial stent; one or more rings of struts at a central portion of the intracranial stent having a diameter that is smaller than a diameter of the one or more rings of struts at the proximal end portion and that is smaller than a diameter of the one or more rings of struts at the distal end portion; one or more rings of struts at a proximal transition portion between the proximal end portion and the central portion; one or more rings of struts at a distal transition portion between the distal end portion and the central portion; and a first ring of connectors connecting a first ring of the one or more rings of struts at the proximal end portion with a second ring of the one or more rings of struts at the proximal transition portion, wherein each connector of the first ring of connectors includes a first curved section, a straight section, and a second curved section.

In some aspects, the techniques described herein relate to an intracranial stent, the one or more rings of struts at the central portion including a first ring and a second ring, wherein the first ring is directly connected to the second ring.

In some aspects, the techniques described herein relate to an intracranial stent, further including: a radiopaque marker including an eyelet portion and a marker engagement strut, wherein the one or more rings of struts at the proximal end portion include a proximal end ring, wherein the marker engagement strut is connected to a crown of the proximal end ring.

In some aspects, the techniques described herein relate to an intracranial stent, wherein the one or more rings of struts at the distal end portion include a distal end ring, and wherein one or more crowns of the distal end ring are chamfered.

In some aspects, the techniques described herein relate to an intracranial stent, wherein the one or more rings of struts at the proximal transition portion gradually increase in diameter from the central portion towards the proximal end portion, and wherein the one or more rings of struts at the distal transition portion gradually increase in diameter from the central portion towards the distal end portion.

In some aspects, the techniques described herein relate to an intracranial stent, wherein each connector of a second ring of connectors at the distal end portion has a smaller radius of curvature than each connector of a third ring of connectors at the central portion.

In some aspects, the techniques described herein relate to an intracranial stent, wherein a distance between the struts at the distal end portion is greater than a distance between the struts at the central portion.

In some aspects, the techniques described herein relate to an intracranial stent, wherein the distal end portion and the proximal end portion are configured to exert a lower radial force than the central portion.

In some aspects, the techniques described herein relate to an intracranial stent, wherein a radial force profile across a length of the intracranial stent is asymmetrical.

In some aspects, the techniques described herein relate to an intracranial stent, wherein the first ring of the one or more rings of struts at the proximal end portion is configured to move towards and away from the second ring of the one or more rings of struts at the proximal transition portion due to extension of the first ring of connectors.

In some aspects, the techniques described herein relate to an intracranial stent, wherein adjacent rings of connectors alternate in direction.

In some aspects, the techniques described herein relate to an intracranial stent, wherein a radial force applied by the central portion is substantially equal along an entire length of the central portion.

In some aspects, the techniques described herein relate to an intracranial stent, wherein the central portion includes a first section and a second section, wherein the first section of the central portion is closer to a distal end of the intracranial stent than the second section of the central portion, and wherein the first section of the central portion exerts a higher radial force than the second section of the central portion.

In some aspects, the techniques described herein relate to an intracranial stent, wherein the proximal end portion includes a first section and a second section, wherein the second section of the proximal end portion is closer to a proximal end of the intracranial stent, and wherein the second section of the proximal end portion exerts a higher radial force than the first section of the proximal end portion.

In some aspects, the techniques described herein relate to an intracranial stent, wherein the distal end portion includes a first section and a second section, wherein the first section of the distal end portion is closer to a distal end of the intracranial stent, and wherein the first section of the distal end portion exerts a higher radial force than the second section of the distal end portion.

In some aspects, the techniques described herein relate to an intracranial stent, wherein the radiopaque marker includes an engagement member that protrudes laterally outward from the marker engagement strut.

In some aspects, the techniques described herein relate to an intracranial stent, further including: a distal end radiopaque marker, wherein a radial thickness of the distal end radiopaque marker is greater than a radial thickness of the one or more rings of struts at the distal end portion.

In some aspects, the techniques described herein relate to an intracranial stent, wherein a radial thickness of the one or more rings of struts at the central portion is greater than the radial thickness of the one or more rings of struts at the distal end portion.

In some aspects, the techniques described herein relate to an intracranial stent, wherein the radial thickness of the distal end radiopaque marker is equal to the radial thickness of the one or more rings of struts at the central portion.

In some aspects, the techniques described herein relate to an intracranial stent, wherein a radial thickness of the one or more rings of struts at the distal transition portion gradually increases from the radial thickness of the one or more rings of struts at the distal end portion to the radial thickness of the one or more rings of struts at the central portion.

In some aspects, the techniques described herein relate to an intracranial stent, wherein the first curved section is abutting the straight section, and wherein the second curved section is abutting the straight section.

In some aspects, the techniques described herein relate to an intracranial stent, further including: a second ring of connectors connecting the first ring of the one or more rings of struts at the proximal end portion with a second ring of the one or more rings of struts at the proximal end portion, wherein each connector of the second ring of connectors includes a first curved section and a second curved section abutting the first curved section.

In some aspects, the techniques described herein relate to an intracranial stent, including: a proximal end portion; a distal end portion; a central portion having a diameter that is smaller than a diameter of the proximal end portion and that is smaller than a diameter of the distal end portion; a plurality of struts, wherein a first group of struts is connected to define a first ring of struts, and wherein a second group of struts is connected to define a second ring of struts; and a plurality of connectors, wherein each connector of the plurality of connectors includes a longitudinal axis, wherein a first group of the plurality of connectors connects the first ring of struts to the second ring of struts.

In some aspects, the techniques described herein relate to an intracranial stent, wherein each connector of the first group of the plurality of connectors is configured to twist about the longitudinal axis.

In some aspects, the techniques described herein relate to an intracranial stent, wherein a distance between the first ring of struts and the second ring of struts is configured to increase and/or decrease as the first group of the plurality of connectors lengthen and shorten, respectively.

In some aspects, the techniques described herein relate to an intracranial stent, wherein a third group of the plurality of struts are connected to define a third ring of struts, wherein the third ring of struts is directly connected to the first ring of struts.

In some aspects, the techniques described herein relate to an intracranial stent, including: a plurality of rings; and a plurality of struts defining the plurality of rings, the plurality of rings including a first ring with a first strut connected to a second strut at a first crown, a third strut connected to the second strut at a second crown, and a fourth strut connected to the third strut at a third crown, wherein the first crown, the second crown, and the third crown are staggered in the longitudinal direction with respect to one another.

In some aspects, the techniques described herein relate to an intracranial stent, further including: a second ring of the plurality of rings, the second ring of the plurality of rings including a fifth strut connected to a sixth strut at a fourth crown, a seventh strut connected to the sixth strut at a fifth crown, and an eighth strut connected to the seventh strut at a sixth crown; and a first ring of connectors connecting the first ring of struts to the second ring of struts, the first ring of connectors including a first connector and a second connector, wherein the first connector extends between the first crown and the fourth crown, wherein the second connector extends between the third crown and the sixth crown, and wherein the first connector is a different shape than the second connector.

In some aspects, the techniques described herein relate to a method of treating an intracranial atherosclerotic stenosis in an intracranial blood vessel, the method including: deploying an intracranial stent in the intracranial blood vessel, the intracranial stent including: a proximal end portion; a distal end portion; a central portion having a diameter that is smaller than a diameter of the proximal end portion and that is smaller than a diameter of the distal end portion; and a first ring of connectors connecting a first ring of struts at the central portion with a second ring of struts at the central portion, wherein each connector of the first ring of connectors includes a first curved section, a straight section, and a second curved section.

In some aspects, the techniques described herein relate to a method of treating an intracranial atherosclerotic stenosis in an intracranial blood vessel, the method including: forming an intracranial stent, the intracranial stent including: a proximal end portion; a distal end portion; a central portion having a diameter that is smaller than a diameter of the proximal end portion and that is smaller than a diameter of the distal end portion; and a first ring of connectors connecting a first ring of struts at the central portion with a second ring of struts at the central portion, wherein each connector of the first ring of connectors includes a first curved section, a straight section, and a second curved section.

In some aspects, the techniques described herein relate to a method of treating an intracranial atherosclerotic stenosis in an intracranial blood vessel, the method including: inserting an intracranial stent into a microcatheter, the intracranial stent including, the intracranial stent including: a proximal end portion; a distal end portion; a central portion having a diameter that is smaller than a diameter of the proximal end portion and that is smaller than a diameter of the distal end portion; and a first ring of connectors connecting a first ring of struts at the central portion with a second ring of struts at the central portion, wherein each connector of the first ring of connectors includes a first curved section, a straight section, and a second curved section.

In some aspects, the techniques described herein relate to an intracranial stent, including: one or more rings of struts at a proximal end portion of the intracranial stent; one or more rings of struts at a distal end portion of the intracranial stent; one or more rings of struts at a central portion of the intracranial stent having a diameter that is smaller than a diameter of the one or more rings of struts at the proximal end portion and that is smaller than a diameter of the one or more rings of struts at the distal end portion; one or more rings of struts at a proximal transition portion between the proximal end portion and the central portion; one or more rings of struts at a distal transition portion between the distal end portion and the central portion; and a first ring of connectors connecting a first ring of the one or more rings of struts at the proximal end portion with a second ring of the one or more rings of struts at the proximal end portion, wherein each connector of the second ring of connectors includes a first curved section and a second curved section abutting the first curved section.

In some aspects, the techniques described herein relate to an intracranial stent, including: one or more rings of struts at a proximal end portion of the intracranial stent; one or more rings of struts at a distal end portion of the intracranial stent; one or more rings of struts at a central portion of the intracranial stent having a diameter that is smaller than a diameter of the one or more rings of struts at the proximal end portion and that is smaller than a diameter of the one or more rings of struts at the distal end portion; one or more rings of struts at a proximal transition portion between the proximal end portion and the central portion; one or more rings of struts at a distal transition portion between the distal end portion and the central portion; and a first ring of connectors connecting a first ring of the one or more rings of struts at the central portion with a second ring of the one or more rings of struts at the central portion, wherein each connector of the first ring of connectors includes a first curved section, a straight section, and a second curved section.

In some aspects, the techniques described herein relate to an intracranial stent, including: one or more rings of struts at a proximal end portion of the intracranial stent; one or more rings of struts at a distal end portion of the intracranial stent; one or more rings of struts at a central portion of the intracranial stent having a diameter that is smaller than a diameter of the one or more rings of struts at the proximal end portion and that is smaller than a diameter of the one or more rings of struts at the distal end portion; one or more rings of struts at a proximal transition portion between the proximal end portion and the central portion; one or more rings of struts at a distal transition portion between the distal end portion and the central portion; and a first ring of connectors connecting a first ring of the one or more rings of struts at the distal end portion with a second ring of the one or more rings of struts at the distal end portion, wherein each connector of the second ring of connectors includes a first curved section and a second curved section abutting the first curved section.

In some aspects, the techniques described herein relate to an intracranial stent, including: one or more rings of struts at a proximal end portion of the intracranial stent; one or more rings of struts at a distal end portion of the intracranial stent; one or more rings of struts at a central portion of the intracranial stent having a diameter that is smaller than a diameter of the one or more rings of struts at the proximal end portion and that is smaller than a diameter of the one or more rings of struts at the distal end portion; one or more rings of struts at a proximal transition portion between the proximal end portion and the central portion; one or more rings of struts at a distal transition portion between the distal end portion and the central portion; and a first ring of connectors connecting a first ring of the one or more rings of struts at the distal end portion with a second ring of the one or more rings of struts at the distal transition portion, wherein each connector of the first ring of connectors includes a first curved section, a straight section, and a second curved section.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts a cross-sectional view of an exemplary intracranial stent, according to one or more embodiments of the present invention.

FIG. 2 depicts a view of an exemplary radiopaque marker, according to one or more embodiments of the present invention.

FIG. 3 depicts a cross-sectional view of an exemplary intracranial stent with a symmetrical radial force profile, according to one or more embodiments of the present invention.

FIG. 4 depicts a cross-sectional view of an exemplary intracranial stent with an asymmetrical radial force profile, according to one or more embodiments of the present invention.

FIG. 5A depicts a cross-sectional view of an exemplary intracranial stent, according to one or more embodiments of the present invention.

FIG. 5B depicts a schematic view of exemplary wall thickness, according to one or more embodiments of the present invention.

FIG. 6A depicts a side view of an exemplary intracranial stent, according to one or more embodiments of the present invention.

FIG. 6B depicts a schematic view of exemplary wall thickness, according to one or more embodiments of the present invention.

FIG. 7 depicts a side view of an exemplary intracranial stent with transition angles, according to one or more embodiments of the present invention.

FIG. 8 depicts a side view of an exemplary intracranial stent with flared rings, according to one or more embodiments of the present invention.

FIG. 9 depicts a side view of an exemplary intracranial stent with a flared end section, according to one or more embodiments of the present invention.

FIG. 10A depicts a cross-sectional view of an exemplary intracranial stent deployed in an intracranial vessel, according to one or more embodiments of the present invention.

FIG. 10B depicts an isometric view of an obstruction after deployment of an intracranial stent, according to one or more embodiments of the present invention.

FIG. 11A depicts a side view of an exemplary intracranial stent, according to one or more embodiments of the present invention.

FIG. 11B depicts a perspective view of an exemplary connector between struts of the intracranial stent of FIG. 11A, according to one or more embodiments of the present invention.

FIG. 11C depicts a perspective view of the connector of FIG. 11B, according to one or more embodiments of the present invention.

FIG. 11D depicts a schematic view of an exemplary intracranial stent in a loaded configuration, according to one or more embodiments of the present invention.

FIG. 12A depicts a side view of an exemplary intracranial stent, according to one or more embodiments of the present invention.

FIG. 12B depicts a perspective view of an exemplary connector between struts of the intracranial stent of FIG. 12A, according to one or more embodiments of the present invention.

FIG. 13A depicts a side view of an exemplary intracranial stent, according to one or more embodiments of the present invention.

FIG. 13B depicts a perspective view of an exemplary crown and pair of struts of the intracranial stent of FIG. 13A, according to one or more embodiments of the present invention.

FIG. 13C depicts a side view of the crown and struts of FIG. 13B, according to one or more embodiments of the present invention.

FIGS. 14A and 14B depict a side view of a portion of an intracranial stent, according to one or more embodiments of the present invention.

FIG. 15 depicts a perspective view of a portion of an intracranial stent being re-sheathed by a microcatheter, according to one or more embodiments of the present invention.

FIG. 16 depicts a two-dimensional projection that represents a portion of an intracranial stent in a loaded configuration, according to one or more embodiments of the present invention.

FIG. 17 depicts a two-dimensional project of a portion of an intracranial stent in the loaded configuration, according to one or more embodiments of the present invention.

FIG. 18A depicts a schematic view of a connector, crowns, and struts cut from tubing material, according to one or more embodiments of the present invention.

FIG. 18B depicts a cross-section view of Section A-A of FIG. 18A, according to one or more embodiments of the present invention.

FIG. 18C depicts a cross-section view of Section B-B of FIG. 18A, according to one or more embodiments of the present invention.

FIG. 18D depicts a schematic view of a connector, crowns, and struts, according to one or more embodiments of the present invention.

FIG. 19 depicts a two-dimensional projection of a portion of an intracranial stent in a collapsed configuration, according to one or more embodiments of the present invention.

### DETAILED DESCRIPTION OF EMBODIMENTS

Both the foregoing general description and the following detailed description are exemplary and explanatory only and are not restrictive of the features, as claimed. As used herein, the terms “comprises,” “comprising,” “has,” “having,” “includes,” “including,” or other variations thereof, are intended to cover a non-exclusive inclusion, such that a process, method, article, or apparatus that comprises a list of elements does not include only those elements, but may include other elements not expressly listed or inherent to such a process, method, article, or apparatus. In this disclosure, unless stated otherwise, relative terms, such as, for example, “about,” “substantially,” and “approximately” are used to indicate a possible variation of ±10% in the stated value. In this disclosure, unless stated otherwise, any numeric value may include a possible variation of ±10% in the stated value.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section.

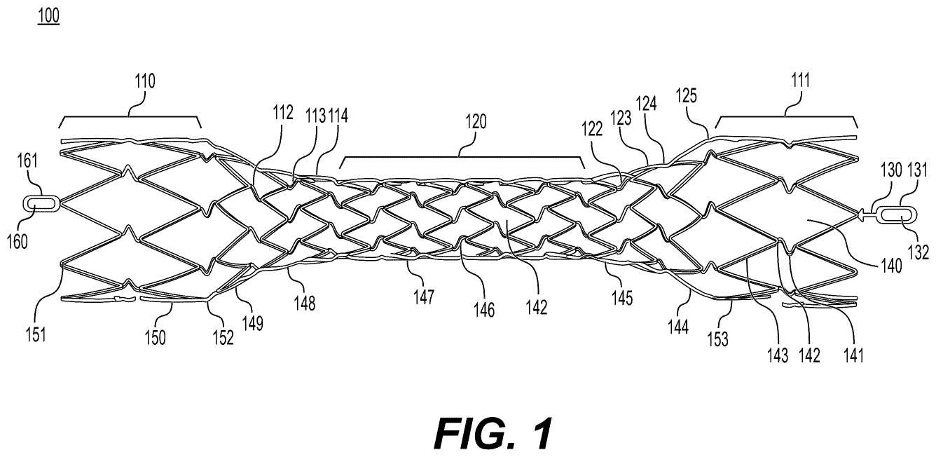

FIG. 1 depicts a cross-sectional view of an exemplary intracranial stent 100, according to one or more embodiments of the present invention. Intracranial stent 100 may include a distal end section 110, a proximal end section 111, and a midsection (e.g., central section) 120. Intracranial stent 100 may be generally dumbbell shaped with a narrowed central portion. For example, distal end section 110 and proximal end section 111 may each be wider than midsection 120. In some embodiments, stent 100 includes a proximal transition portion 124 and a distal transition portion 149.

The intracranial stent 100 may comprise a series of rings connected to one another by connectors. The rings may include a plurality of struts in a “zig-zag” pattern, and the plurality of struts may include crowns at the end sections of the struts. One ring of struts may have 6 struts, 8 struts, 10 struts, 12 struts, 16 struts 18 struts, or 20 struts. The connectors may connect the rings to one another at the crowns of the struts, as will be further described below with respect to FIG. 1.

The space between adjacent rings of struts and/or the space between adjacent struts may be smaller at midsection 120 than distal end section 110 or proximal end section 111. That is, as depicted in FIG. 1, midsection cell area 162 is smaller than proximal cell area 140. In other words, the distance between adjacent rings of struts is smaller at the midsection 120 of intracranial stent 100 than the distance between adjacent rings of struts at the distal end section 110 or at the proximal end section 111. The smaller cell area of midsection cell area 162 allows intracranial stent 100 to scaffold plaque and prevent embolization where pieces of plaque break off (e.g., prevent the struts of midsection 120 of intracranial stent 100 from cutting into the plaque). Additionally, the large cell area of proximal cell area 140 (and similarly the cell area at distal end section 110) minimizes obstructing natural blood flow into vessels branching off from the vessel intracranial stent 100 is installed in while also minimizing unnatural blood flow (e.g., high shear flow, turbulent flow, etc.) into the side vessels.

The connectors at different sections of intracranial stent 100 may be different shapes. For example, the connectors may start as a “V” shape or a check mark shape at distal end section 110 and proximal end section 111 and transition to more of an “S” shape closer to midsection 120. Connectors 112 in a transition section between distal end section 110 and midsection 120 may be elongated (e.g., more “S” shaped than the connectors at distal end section 110 or proximal end section 111). In this way, the connectors in the distal end section 110 or the proximal end section 111 may be curved at a greater angle, or radius of curvature, than the connectors at midsection 120. Connectors in the distal end section 110 or the proximal end section 111 may include a first curved portion adjacent to a second curved portion (e.g., creating the “V” shape).

The shape of the connectors may be optimized for flexibility in the collapsed and expanded configurations of intracranial stent 100. The flexibility may further be optimized by alternating the direction of the connectors of intracranial stent 100 along the length of intracranial stent 100. That is, adjacent connectors may be flipped vertically, as shown in FIG. 1, with respect to one another. For example, connector 112 is higher at the left end than the right end, while connector 113 (which is directly adjacent to connector 112) is higher at the right end than the left end. During deployment of intracranial stent 100, the tip of a microcatheter may move along the surface of intracranial stent 100. The intracranial stent 100 (e.g., connectors and struts of intracranial stent 100) may apply a torque onto the microcatheter. Alternating the direction of the connectors changes the direction of the torque and may substantially reduce and/or eliminate the torque applied to the microcatheter when deploying intracranial stent 100. The flexibility or stiffness of stent 100 may be controlled by the connector shapes which are designed to transition the stiffness gradually along the length of the stent. Thus, the stiffness of the midsection 120 may gradually change to the stiffness of the proximal section 111 or end section 110. This gradual transition may reduce the risk of the stent 100 kinking when deployed in a curved vessel.

Each connector connects struts from one ring to another ring. As depicted in FIG. 1, a ring of struts 114 may be connected to another ring of struts 148 via connectors 113. The transition portions 124, 149 between distal end section 110 and midsection 120, and between proximal end section 111 and midsection 120, may include flared rings of struts, such as ring of struts 144. Struts 114 and struts 148 may also be flared. Ring of struts 114 and/or ring of struts 145 may be configured to apply a medium amount radial force. Ring of struts 148 and/or ring of struts 123 may be configured to apply a low amount of radial force, as further described below. In general, midsection 120 may be configured to apply high radial force, such as ring of struts 147, while distal end section 110 and proximal end section 111 are configured to apply low radial force. Struts 143 within the same ring may be connected at a crown 142 while struts 143 in different rings may be connected by connectors, such as connector 112, connector 113, or connector 122. A connector 125 may be between a transition section of intracranial stent 100 and proximal end section 111 of intracranial stent 100. A connector 146 may be within midsection 120 of intracranial stent 100. A connector 152 may be between a transition section of intracranial stent 100 and distal end section 110. A connector 141 may be within proximal end section 111 of intracranial stent 100.

As discussed previously, the connectors connect the crowns 142 of struts 143 of different rings. Struts 143 or struts 150 may be tapered. Stent 100, including struts 143, may be made of nitinol, stainless steel, titanium, cobalt chrome or any other suitable metallic and/or biodegradable material. The stent configuration described in FIG. 1 may be a self-expanding stent, while in other embodiments the stent may be expanded by a balloon, A final ring of struts at distal end section 110 may include distal end crowns 151 and a final ring of struts at proximal end section 111 may include proximal end crowns 126. Distal end section 110 may include a distal radiopaque marker 161 extending away from distal end section 110. As depicted in FIG. 1, distal radiopaque marker 161 may extend from one of the distal end crowns 151. Distal radiopaque marker 161 may form an eyelet directly connected to distal end crowns 151 in which radiopaque material 160 is present. Proximal end section 111 may include a proximal radiopaque marker 131 forming an eyelet extending away from proximal end section 111. More specifically, proximal radiopaque marker 131 may extend from one of proximal end crowns 126. Proximal radiopaque marker 131 may include an eyelet portion similar to distal radiopaque marker 161, marker 131 including radiopaque marker 132. A marker engagement strut 130 may connect the proximal radiopaque marker 200 to one of the distal end crowns 151.

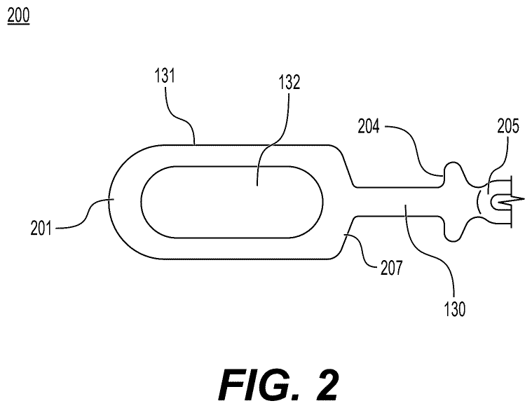

FIG. 2 depicts a side view of an exemplary radiopaque marker 200, according to one or more embodiments of the present invention. Radiopaque marker 200 may be similar to proximal radiopaque marker 132. Proximal radiopaque marker 200 may include an eyelet portion 201 with a radiopaque material 203 and a marker engagement strut 202 that extends away from proximal end crown 205. Marker engagement strut 202 may include an engagement member 204 that flares out on both sides of marker engagement strut 202. Engagement member 204 may protrude from marker engagement strut 202. The marker engagement strut 202 may interlock with a delivery shaft when a stent connected to proximal radiopaque marker 200 is being deployed through a microcatheter. For example, the delivery shaft may engage with engagement member 204 during delivery through the microcatheter as the stent is pushed distally by the shaft, and abut contact face 207, partially surrounding marker engagement strut 202, during re-sheathing as the delivery shaft keeps the stent static or pulls the stent proximally. Proximal radiopaque marker 200 may interlock with the delivery shaft to assist with pushing the intracranial stent through the microcatheter and/or for retraction during re-sheathing.

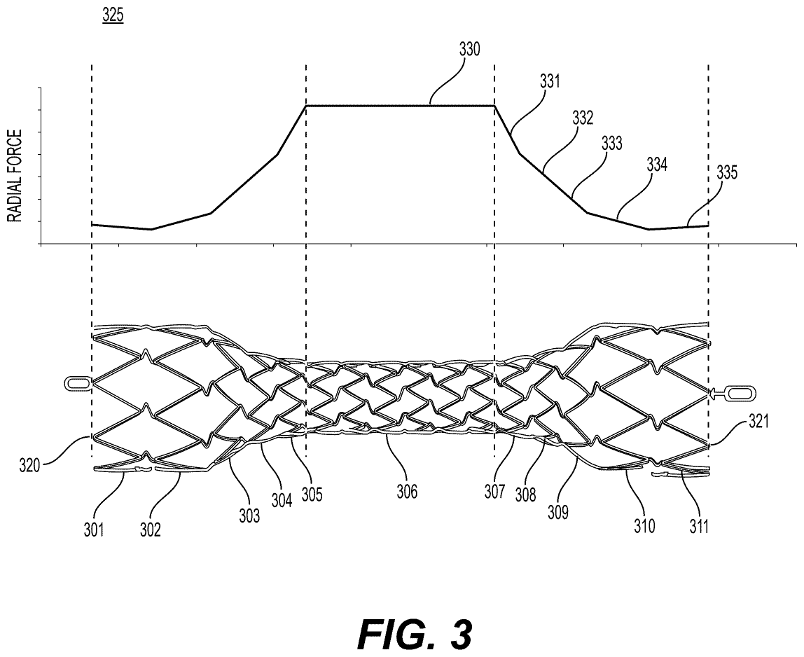

FIG. 3 depicts a side view of an exemplary intracranial stent 300 with a symmetrical radial force profile, according to one or more embodiments of the present invention. Intracranial stent 300 may be similar to intracranial stent 100. Intracranial stent 300 may include a distal anchor ring of struts 301, a distal ring of tapered struts 302, a transition ring of struts 303, a low radial force ring of struts 304, a medium radial force ring of struts 305, a high radial force ring of struts 306, a medium radial force ring of struts 307, a low radial force ring of struts 308, a transition ring of struts 309, a proximal ring of tapered struts 310, a proximal anchor ring of struts 311, a distal end crown 320, and a proximal end crown 321. Radial force profile 325 may show a graph of the radial force needed to compress and/or deform intracranial stent 300 along the length of intracranial stent 300 at a given diameter. It should be appreciated that the terms low radial force, medium radial force, and high radial force used herein are describing the forces relative to each other and not representative of an actual amount of force.

The radial force may be measured by any suitable method. For example, radial force profile 325 may be measured using a radial force compression station with an iris (e.g., according to ISO 25539 or ASTM F3067). Intracranial stent 300 may be inserted into the station, and the force of the stent against the iris while the iris is opened and closed may be measured. In another example, radial force profile 325 may be measured by determining a hoop force that deforms intracranial stent 300 and converting the hoop force to radial force. After measuring the hoop or radial force, a ring or multiple rings of struts of intracranial stent 300 may be removed and the force is measured again. In this way, the hoop force or radial force of each ring of struts can be determined to generate radial force profile such as radial force profile 325.

Radial force profile 325 may include a midsection force portion 330, a medium force ring force portion 331, a low force ring force portion 332, a transition ring force portion 333, a tapered ring force portion 334, and an anchor ring force portion 335. As is illustrated in FIG. 3, midsection force portion 330 corresponds to the radial force at high radial force ring of struts 306, medium force ring force portion 331 corresponds to medium radial force ring of struts 307, low force ring force portion 332 corresponds to low radial force ring of struts 308, transition ring force portion 333 corresponds to transition ring of struts 309, tapered ring force portion 334 corresponds to proximal ring of tapered struts 310, and anchor ring force portion 335 corresponds to proximal anchor ring of struts 311. It should be appreciated that radial force profile 325 is symmetric on each side of midsection force portion 330. Therefore, the radial force sections described in relation to the proximal side of intracranial stent 300 are symmetrical to radial force sections corresponding to the distal side of intracranial stent 300. The maximum radial force may be at the midsection of intracranial stent 300 (e.g., high radial force ring of struts 306).

As shown with radial force profile 325, the midsection of intracranial stent 300 (e.g., midsection force portion 330) is configured to exert a higher radial force than at the distal or proximal section ends (e.g., anchor ring force portion 335). The low radial force seen in radial force profile 325 corresponding to the force exerted by the proximal and distal end sections of intracranial stent 300 may be atraumatic to the vessel. Thus, when deployed, intracranial stent 300 applies a larger radial force against plaque to open up the vessel than the force applied directly to the vessel wall. The radial force may be profiled to reduce gradually when going from high radial force ring of struts 306 at the midsection of intracranial stent 300 towards proximal anchor ring of struts 311. However, the radial force may increase from tapered ring force portion 334 to anchor ring force portion 335. Proximal anchor ring of struts 311 and distal anchor ring of struts 301 may have an increase in radial force to anchor intracranial stent 300 in the vessel.

Flared sections of intracranial stent 300 (e.g., medium radial force ring of struts 307, low radial force ring of struts 308, and transition ring of struts 309) may exert a reduced radial force to not apply point loading to the vessel wall. The flared sections may also improve blood flow dynamics and increase laminar flow. As explained previously, the cell areas gradually increase from the midsection of intracranial stent 300 to the ends of intracranial stent 300 to optimize necessary scaffolding. The inclusion of tapered struts along the length of intracranial stent 300 (e.g., proximal ring of tapered struts 311) may help fine tune the radial force. When tested in a 1.0 mm lumen diameter, the self-expanding outward radial force/length of the stent 100, in at least some embodiments, varies from 0.03 N/mm up to 1.0 N/mm, when calculated per unit length.

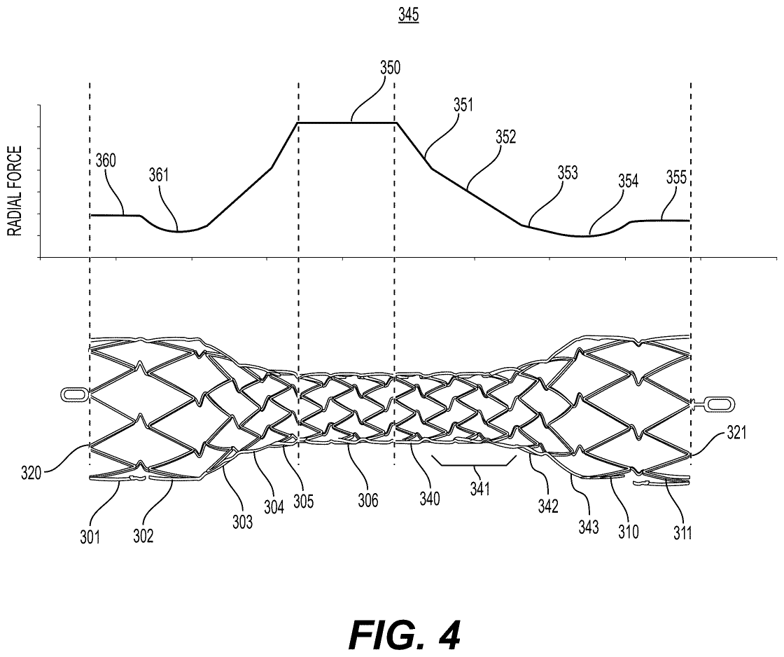

FIG. 4 depicts a side view of an exemplary intracranial stent with an asymmetrical radial force profile, according to one or more embodiments of the present invention. Intracranial stent 336 may be similar to intracranial stent 300. For example, intracranial stent 336 may include distal anchor ring of struts 301, distal ring of tapered struts 302, transition ring of struts 303, low radial force ring of struts 304, medium radial force ring of struts 305, high radial force ring of struts 306, proximal ring of tapered struts 310, proximal anchor ring of struts 311, distal end crown 320, and proximal end crown 321. Additionally, near the proximal side of the midsection and transition section, intracranial stent 336 may include a medium radial force ring of struts 340, decreasing radial force rings 341, low radial force ring of struts 342, and transition ring of struts 343. Radial force profile 345 may show a graph of the radial force that intracranial stent 336 exerts. For example, radial force profile 345 corresponds to the amount of outward force that the intracranial stent 336 is capable of exerting against a structure such as a stenosis, at a given lumen diameter, such as diameters between 1.0 and 4.0 mm.

Radial force profile 345 may include a midsection force portion 350, a medium force ring force portion 351, a decreasing force ring force portion 352, a low force ring force portion 353, a tapered ring force portion 354, an anchor ring force portion 355, an anchor ring force portion 360, and a tapered ring force portion 361. As is illustrated in FIG. 4, midsection force portion 350 corresponds to the radial force at high radial force ring of struts 306, medium force ring force portion 351 corresponds to medium radial force ring of struts 340, decreasing force ring force portion 352 corresponds to decreasing radial force rings 341, low force ring force portion 353 corresponds to low radial force ring of struts 342, tapered ring force portion 354 corresponds to proximal ring of tapered struts 310, anchor ring force portion 355 corresponds to proximal anchor ring of struts 311, anchor ring force portion 360 corresponds to distal anchor ring of struts 301, and tapered ring force portion 361 corresponds to distal ring of tapered struts 302. The maximum radial force may be at midsection force portion 350 while the minimum radial force may be at tapered ring force portion 361 and/or tapered ring force portion 354. The ends of radial force profile 345 (e.g., anchor ring force portion 360 and anchor ring force portion 355) may have a greater radial force than tapered ring force portion 361 and tapered ring force portion 354. Radial force profile 345 may increase more rapidly from anchor ring force portion 360 to midsection force portion 350 than radial force profile 345 decreases from midsection force portion 350 to anchor force ring portion 355. In other words, the rate of decrease of the radial force on the proximal side of intracranial stent 336 may be more gradual than the rate of increase of the radial force on the distal side of intracranial stent 336. In some examples, the plaque within a vessel may have a non-uniform distribution. The plaque may have a larger loading in the distal half of the stenosis. Consequently, a larger radial force in the distal half than in the proximal half may be desired. Therefore, radial force profile 345 may better reflect the preferred radial force profile for a stent deployed in a vessel with non-uniformly distributed plaque.

Midsection force portion 350 may be a first section of the midsection of intracranial stent 336 that is a higher radial force than a second section of the midsection of intracranial stent 336. Anchor ring force portion 360 may be a first section of the distal end section of intracranial stent 336 that is a higher radial force than a second section (e.g., tapered ring force portion 361) of the distal end section of intracranial stent 336. A first section (e.g., tapered ring force portion 354) of the proximal end section of intracranial stent 336 may be a lower radial force than a second section (e.g., anchor ring force portion 355) of the proximal end of intracranial stent 336.

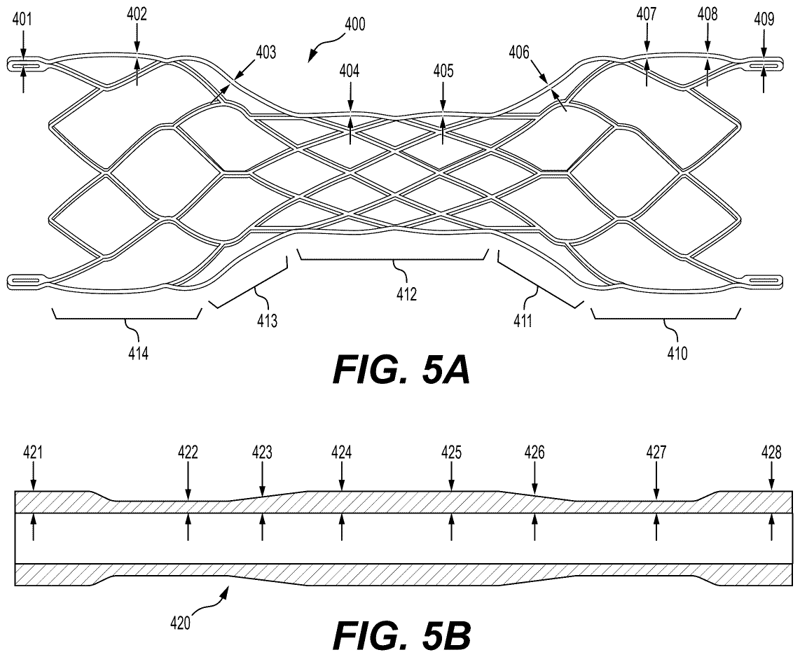

FIG. 5A depicts a side view of an exemplary intracranial stent, according to one or more embodiments of the present invention. Intracranial stent 400 may include a proximal end section 410, a proximal transition section 411, a midsection 412, a distal transition section 413, and a distal end section 414. Intracranial stent 400 may include distal marker 401, anchor ring 402, anchor ring 408, and proximal marker 409. As will be further described with respect to FIG. 5B, the wall thickness of intracranial stent 400 may vary throughout the length of intracranial stent 400.

FIG. 5B depicts a schematic view of exemplary wall thickness 420, according to one or more embodiments of the present invention. Thickness 420 may represent the thickness of walls formed from a tube of material used to form intracranial stent 400. For example, intracranial stent 400 may be laser cut from this tube of material. The tube may be made of nitinol, stainless steel, titanium, or any other suitable material. The thickness 420 may vary throughout the length of stent 400, as shown in FIG. 5B. Thickness 420 of stent 400 may include a wall thickness 421 that corresponds to distal marker 401 and anchor ring 402 of intracranial stent 400, a wall thickness 422 that corresponds to distal end section 414, a wall thickness 423 that corresponds to distal transition section 413, a wall thickness 424 that corresponds with midsection 412, a wall thickness 426 that corresponds with proximal transition section 411, a wall thickness 427 that corresponds with proximal end section 410 and a wall thickness 428 that corresponds with anchor ring 408 and proximal marker 409.

Intracranial stent 400 may be produced by laser cutting a pattern from raw material (e.g., the above-described tubing) and then expanding the pattern onto a series of mandrels to achieve the finished shape of intracranial stent 400. The resulting unfinished intracranial stent may then be annealed and chemically treated to produce the final surface finish and wall thickness of intracranial stent 400.

To vary thickness along length of intracranial stent 400, the material tubing may be ground down before laser cutting, or the surface may be laser-etched during the laser cutting process to reduce the wall thickness in desired areas. Reducing the wall thickness from wall thickness 424 to wall thickness 422 or wall thickness 427 may reduce the radial force in those sections of intracranial stent 400 and increase flexibility of those sections. Wall thickness 421 of anchor ring 402 may be greater than wall thickness 422 to ensure stable anchoring of the stent in the vessel. Wall thickness 421 of distal marker 401 may also provide improved radiopaque marker retention. Similarly, wall thickness 428 of anchor ring 408 and proximal marker 409 may be greater than wall thickness 427 to ensure precise anchoring in the vessel and radiopaque marker retention.

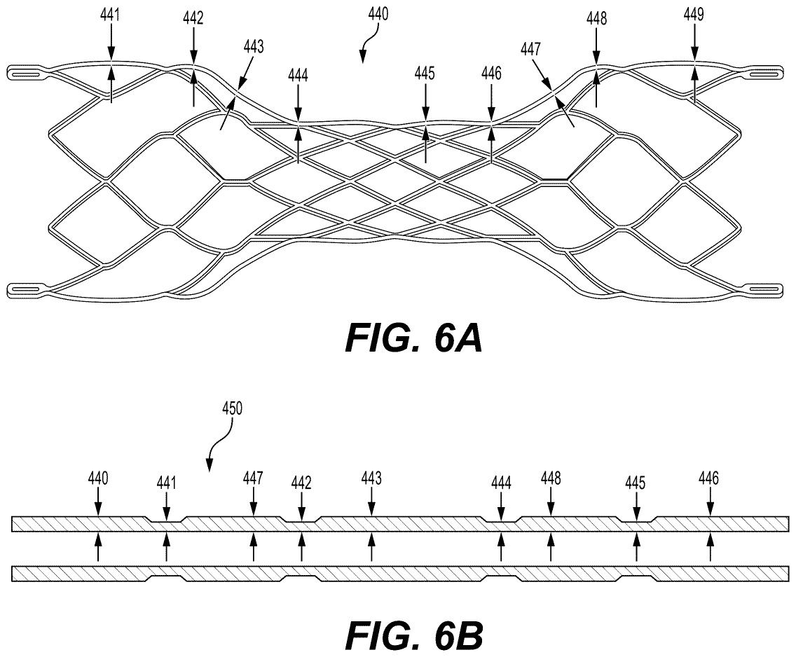

FIG. 6A depicts a side view of an exemplary intracranial stent, according to one or more embodiments of the present invention. Intracranial stent 440 may be similar to intracranial stent 400. For example, intracranial stent 440 may include proximal end section 410, proximal transition section 411, midsection 412, distal transition section 413, and distal end section 414. As will be further described with respect to FIG. 6B, the wall thickness of intracranial stent 440 may vary throughout the length of intracranial stent 440.

FIG. 6B depicts a schematic view of exemplary wall thickness 450, according to one or more embodiments of the present invention. Similar to thickness 420 for intracranial stent 400, thickness 450 may represent the thickness of walls formed from a tube of material used to form intracranial stent 440 in the same ways described previously with respect to FIG. 5B. Thickness 450 may include a wall thickness 441 that corresponds to distal end section 414, a wall thickness 442 that corresponds to an area of intracranial stent 440 where there is curvature between distal end section 414 and distal transition section 413, a wall thickness 443 that corresponds to distal transition section 413, a wall thickness 444 that corresponds to an area of intracranial stent 440 where there is curvature between distal transition section 413 and midsection 412, a wall thickness 445 that corresponds to midsection 412, a wall thickness 446 that corresponds to an area of intracranial stent 440 where there is curvature between midsection 412 and proximal transition section 411, a wall thickness 447 that corresponds to proximal transition section 411, a wall thickness 448 that corresponds to an area of intracranial stent 440 where there is curvature between proximal transition section 411 and proximal end section 410, and a wall thickness 449 that corresponds to proximal end section 410. Therefore, the wall thickness of intracranial stent 440 may be reduced between distal end section 414 and distal transition section 413, distal transition section 413 and midsection 412, midsection 412 and proximal transition section 411, and proximal transition section 411 and proximal end section 410 while the wall thickness of distal end section 414, distal transition section 413, midsection 412, proximal transition section 411, and proximal end section 410 may be kept substantially similar. This change in wall thickness may impact radial force and flexibility as previously described with respect to FIG. 5B.

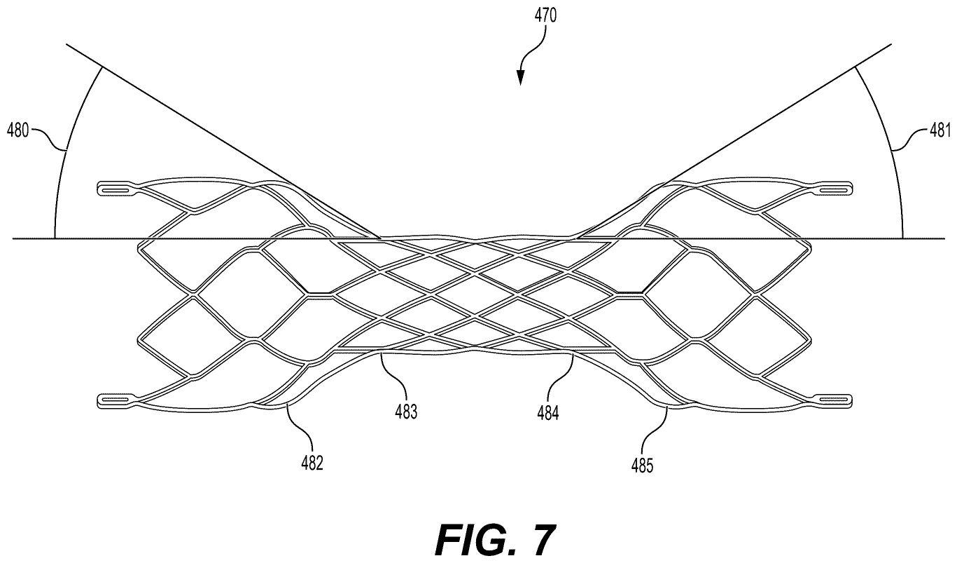

FIG. 7 depicts a side view of an exemplary intracranial stent with a transition angle, according to one or more embodiments of the present invention. Intracranial stent 470 may be similar to intracranial stent 400 or intracranial stent 440. For example, intracranial stent 470 may include proximal end section 410, proximal transition section 411, midsection 412, distal transition section 413, and distal end section 414. Intracranial stent 470 may include curved struts 482 between distal end section 414 and distal transition section 413, curved struts 483 between distal transition section 413 and midsection 412, curved struts 484 between midsection 412 and proximal transition section 411, and curved struts 485 between proximal transition section 411 and proximal end section 410.

A distal transition angle 480 may be the angle at which intracranial stent 470 flares out from midsection 412 towards distal end section 414. Similarly, a proximal transition angle 481 may be the angle at which intracranial stent 470 flares out from midsection 412 towards proximal end section 410. That is, distal transition angle 480 may be the angle between a centerline of the intracranial stent 470 and distal transition section 413 while proximal transition angle 481 may be the angle between the centerline of intracranial stent 470 and proximal transition section 411. Distal transition angle 480 may be the same angle as proximal transition angle 481. In some examples, distal transition angle 480 is a different angle than proximal transition angle 481 (e.g., angle 481 may be larger than angle 480, or angle 480 may be larger than angle 481). The curvature (e.g., curved struts 482, curved struts 483, curved struts 484, and curved struts 485) and angles (e.g., distal transition angle 480 and proximal transition angle 481) may be selected to promote laminar flow in the vessel and optimize blood shear rates.

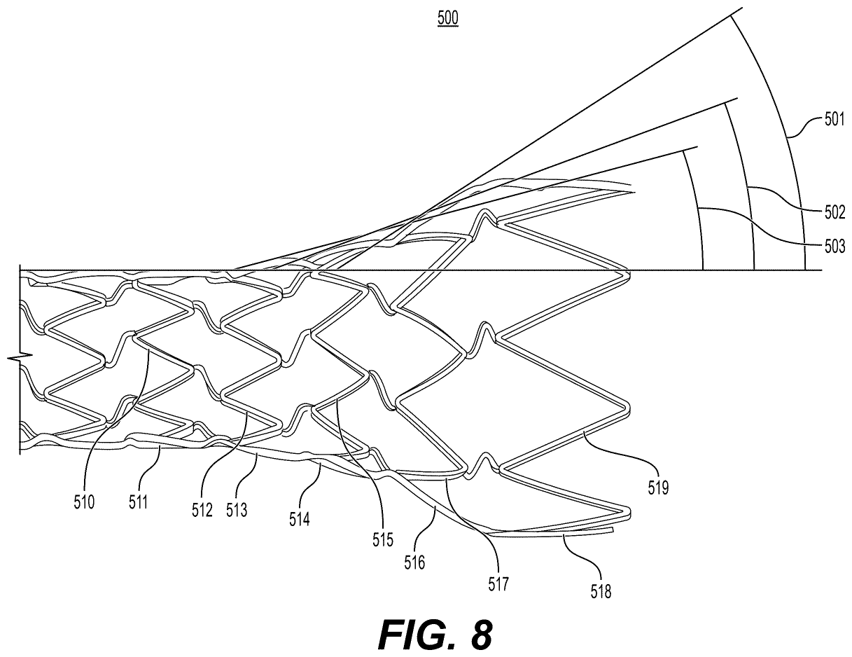

FIG. 8 depicts a side view of an exemplary intracranial stent with flared rings, according to one or more embodiments of the present invention. Specifically, FIG. 8 depicts one side of an intracranial stent 500, such as the proximal side. Intracranial stent 500 may be similar to any of the previous intracranial stents described herein. Intracranial stent 500 may include high radial force struts 510, high radial force rings 511, medium radial force struts 512, medium radial force rings 513, low radial force struts 514, low radial force rings 515, transition section struts 516, transition section rings 517, end section struts 518, and end section rings 519. Transition section flare angle 501 may be the angle at which the transition section of intracranial stent 500 flares out. For example, transition section flare angle 501 may be the angle between a centerline of intracranial stent 500 and transition section struts 516. Similarly, low radial force struts flare angle 502 may be the angle between the centerline of intracranial stent 500 and low radial force struts 514, and medium radial force struts flare angle 503 may be the angle between the centerline of intracranial stent 500 and medium radial force struts 512. As illustrated in FIG. 8, the angles of the flared struts from the midsection of intracranial stent 500 towards the proximal or distal end of intracranial stent 500 gradually increase, in a discrete manner (e.g., with the angle of inclination increasing in a stepwise manner, as opposed to a continuous manner that would be formed by a curved structure). Transition section flare angle 501 may be larger than low radial force struts flare angle 502, and low radial force struts flare angle 502 may be larger than medium radial force struts flare angle 503. The gradual increase in flare angle may promote laminar flow and optimize (e.g., reduce) blood shear rates to below 250 Pa. Transition section flare angle 501 may vary from 10° to 50°. Preferably, transition section flare angle 501 may be in the range of 20° to 40°. Low radial force struts flare angle 502 may vary from 5° to 30° but may preferably be in the range of 10° to 20°. Medium radial force struts flare angle 503 may vary from 2° to 20° but may preferably be in the range of 5° to 15°.

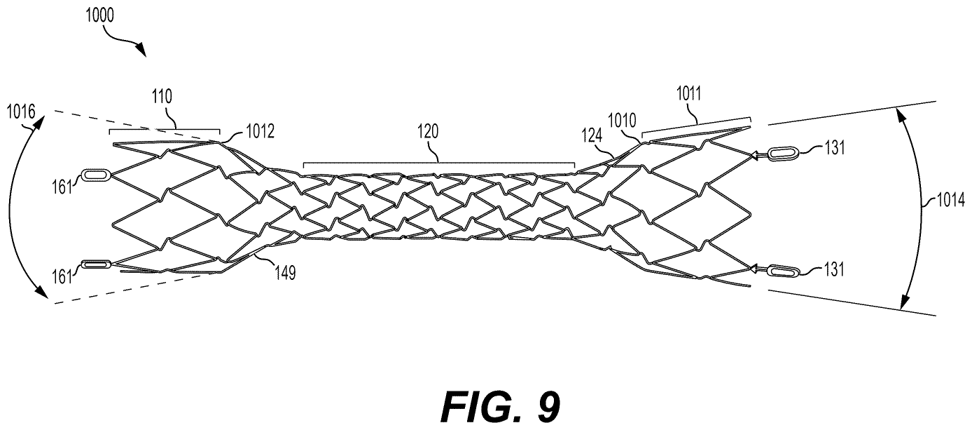

FIG. 9 depicts a side view of an exemplary intracranial stent 1000 with a flared end section, according to one or more embodiments of the present invention. Intracranial stent 1000 may be similar to intracranial stent 100, intracranial stent 300, etc. For example, intracranial stent 1000 may include distal end section 110, proximal end section 1011, and midsection (e.g., central section) 120. Intracranial stent 1000 may also include proximal transition portion 124 and distal transition portion 149. Distal end section 110 may include distal radiopaque marker 161 extending away from distal end section 110. Proximal end section 1011 may include proximal radiopaque marker 131 forming an eyelet extending away from proximal end section 1011. Intracranial stent 1000 may have a proximal transition portion 1010 between proximal transition portion 124 and proximal end section 1011. Intracranial stent 1000 may have a distal transition portion 1012 between distal transition portion 149 and distal end section 110.

A proximal end angle 1014 may be the angle at which intracranial stent 1000 flares out from proximal transition portion 1010 along proximal end section 1011. As represented with dashed lining in FIG. 9, intracranial stent 1000 may further include a distal end angle 1016 which may be the angle at which intracranial stent 1000 flares out from distal transition portion 1012 along distal end section 110. In some examples, proximal end angle 1014 is a different angle than distal end angle 1016 (e.g., angle 1014 may be larger than distal end angle 1016, or distal end angle 1016 may be larger than angle 1014). In some other examples, proximal end angle 1014 may be the same angle as distal end angle 1016. Angle 1014 and distal end angle 1016 may promote laminar flow and optimize (e.g., reduce) blood shear rates to below 250 Pa. Angle 1014 and distal end angle 1016 may gradually increase, in a continuous or discrete manner (e.g., with the angle of inclination increasing in a stepwise manner). Angle 1014 and distal end angle 1016 in the range of about 1° to about 50°, in the range of about 5° to about 30°, or in the range of about 10° to about 20°. In some aspects, angle 1014 and distal end angle 1016 may further be about 15°, about 10°, or about 20°.

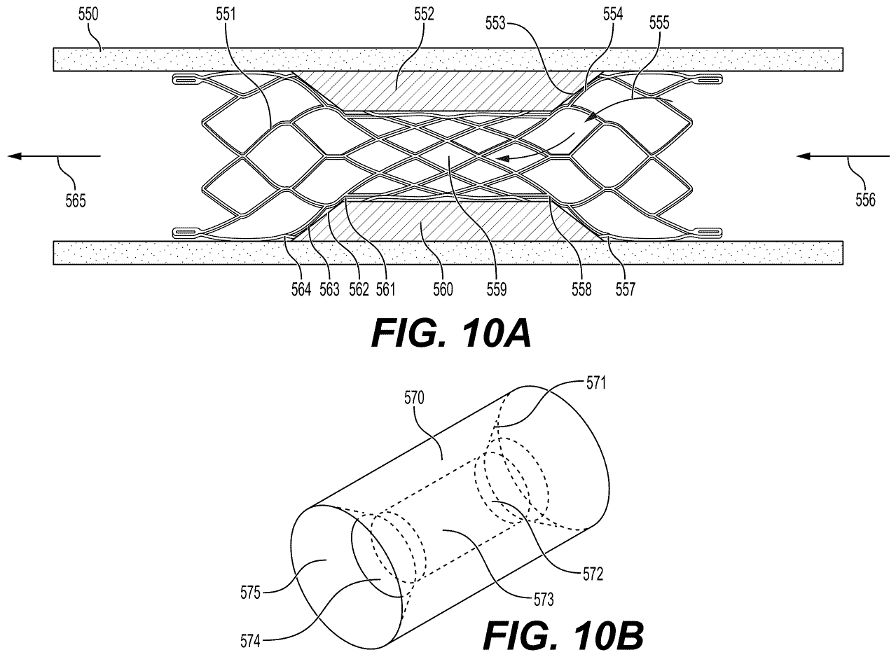

FIG. 10A depicts a cross-sectional view of an exemplary intracranial stent deployed in a previously-obstructed vessel, a vessel containing a stenosis, according to one or more embodiments of the present invention. Intracranial stent 540 similar to any of the previous intracranial stents described herein. Intracranial stent 540 may abut artery wall 550 and obstruction 552 (e.g., a stenosis containing plaque). Specifically, a distal end section 551 and proximal end section 553 may abut artery wall 550 while a midsection 555 may abut obstruction 552. Blood flow 556 in the artery may flow from proximal end section 553 towards distal end section 551. Obstruction 552 may extend from artery wall 550 and, after deployment of stent 540, may be substantially cylindrical with slanted leading and trailing faces. As depicted in FIG. 10A, obstruction 552 may gradually decrease in length when moving away from artery wall 550 towards the center of the artery. Intracranial stent 540 may be designed such that, on deployment in the stenosis in the vessel, the lumen surrounded by the obstruction 552 is dilated to restore blood flow. The expansion of stent 540 may cause the stenosis lumen to dilate and the obstruction 552 may fill the annulus space around midsection 555. The annulus space of stent 540 may be designed to accommodate the volume of the stenosis with minimal pressure applied to the wall of artery 550. FIG. 10A may illustrate this configuration for an obstruction 552 such as a concentric stenosis in artery 550; however, the design of stent 540 may allow the annulus space around midsection 555 to also accommodate eccentric stenoses or obstructions. On deployment of stent 540, the stenosis may conform to the shape and angle of the proximal transition section 572 and distal transition section 574. Stent 540 may be designed so that section 572 promotes laminar blood flow 556 from a non-diseased artery through the stenosis to the distal side.

FIG. 10B depicts an isometric view of an obstruction after deployment of an intracranial stent, according to one or more embodiments of the present invention. Obstruction 570 (e.g., a stenosis containing plaque) may correspond to obstruction 552 depicted in FIG. 10A. More specifically, obstruction 570 may be shaped the way it is because of the stent opening the stenosis due to the above-described radial force distributions. Obstruction 570 may include a proximal leading surface 571, an edge 572 from the deployment of the stent, a flow lumen 573 through obstruction 570, an edge 574 from the deployment of the stent, and a distal trailing surface 575. Proximal leading surface 571 and distal trailing surface 575 may take the shape of the transition sections of the stent (e.g., intracranial stent 540) to promote laminar flow, creating edge 572 and edge 574. The annular space around the midsection (e.g., midsection 555) may be sized such that it accommodates the volume of the plaque with minimal vessel wall distension or expansion. Therefore, the risk of the obstruction 570 being moved into side branches and vessel dissection may be reduced.

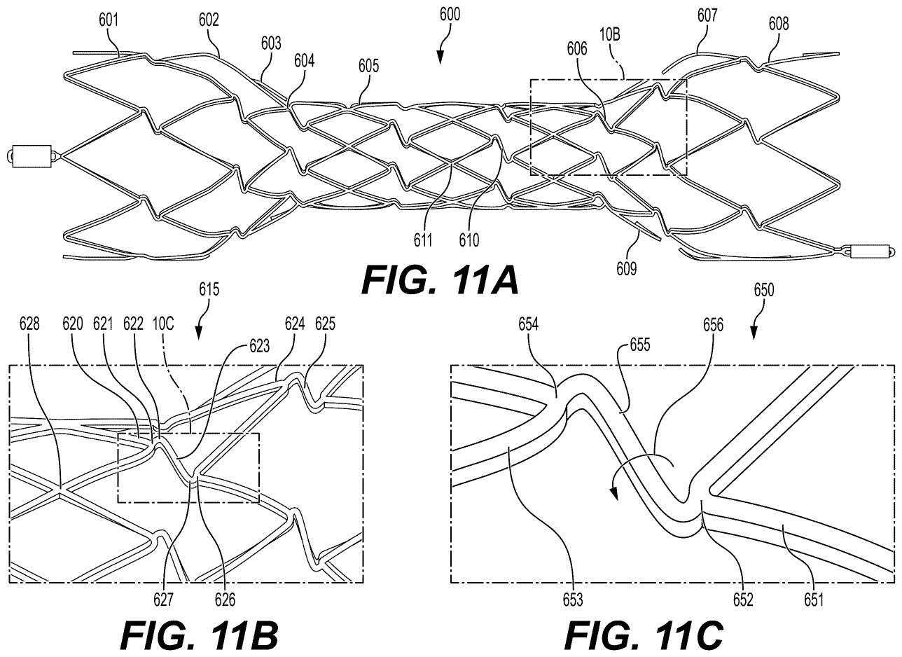

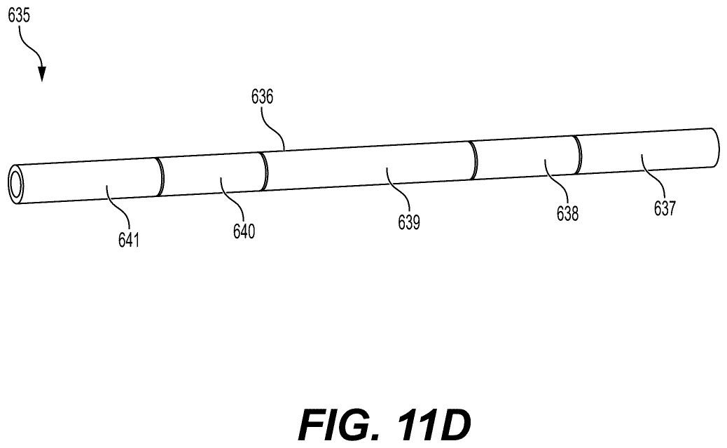

FIG. 11A depicts a side view of an exemplary intracranial stent, according to one or more embodiments of the present invention. Intracranial stent 600 may be similar to any of the previous intracranial stents described herein. Intracranial stent 600 may include end section 601, curved struts 602, transition section 603, connector 604, midsection 605, connector 606, curved struts 607, end section 608, transition section 609, connector 610, and short connector 611. Short connector 611 may be different from connector 610 or other connectors of intracranial stent 600 as connector 611 may directly connect the crowns of two adjacent struts instead of coupling a segment between the two crowns.

FIG. 11B depicts a perspective view of an exemplary connector between struts of the intracranial stent of FIG. 11A, according to one or more embodiments of the present invention. Connector 606 connects a crown 621 of a midsection strut 620 to a crown 626 of a transition section strut 624. Connector 625 may be a connector between crowns of transition section struts and short connector 628 may directly connect crowns of midsection struts from different rings to one another. Connector 606 may include a curved joint (e.g., portion) 622, a straight portion 623, and a curved joint (e.g., portion) 627. Curved joint 622 may be adjacent to straight portion 623, and curved joint 627 may be adjacent to straight portion 623. Straight portion 623 may include a longitudinal axis that defines the longitudinal axis of connector 606.

FIG. 11C depicts a perspective view of the connector of FIG. 11B, according to one or more embodiments of the present invention. Straight portion 623 may be twisted in a direction 656 upon expansion of stent 600. Straight portion 623 may form a torque hinge between midsection strut 620 and transition section strut 624. When in the collapsed configuration, intracranial stent 600 may be generally cylindrical for delivery through a microcatheter to the target deployment site. To facilitate formation of different diameters of the end sections of intracranial stent 600 post deployment, transition section 603 and transition section 609 may be hinged at either end. The connectors between transition sections 603, 609 and midsection 605 (e.g., connector 606) may act as a torque hinge twisting along the length (e.g., a longitudinal axis) of the connector. This may allow the transitions sections 603, 609 to flare and wrap into a cylindrical configuration when collapsed. Additionally, the twist in the connectors may allow intracranial stent 600 to be deployed in an eccentric obstruction with one side of intracranial stent 600 expanded and the other side (e.g., one side opposing the other side in a radial direction) of intracranial stent 600 matching the cylindrical vessel wall. The twist along the longitudinal axis of the connectors may occur anywhere there is a difference in the flare angle of the ring of struts. FIG. 11B illustrates portion 623 between the midsection 605 and transition section 609. Similarly, connector 604 may have a partial twist along the length. This twist may also occur to a lesser extent in the connectors between the flared strut rings 513, 515, 517 and 519 described in FIG. 8.

FIG. 11D depicts a schematic view of an exemplary intracranial stent in a loaded configuration (e.g., a configuration where the intracranial stent is loaded in a microcatheter), according to one or more embodiments of the present invention. Loaded intracranial stent 635 may have a diameter 636 measured at a midsection 639 of loaded intracranial stent 635. Loaded intracranial stent 635 may include an end section 637, a transition section 638, midsection 639, a transition section 640, and an end section 641 that each have the same diameter 636 as midsection 639. Loaded intracranial stent 635 may be deployed into an artery vessel in this configuration. The ability of the connectors to act as a torque hinge may allow the transition sections 638, 640 and the end sections 637, 641 to collapse uniformly into a cylindrical configuration. This uniform wrapping in a cylindrical configuration with straight struts aligning with the stent longitudinal axis may facilitate a reduced force delivery through the microcatheter.

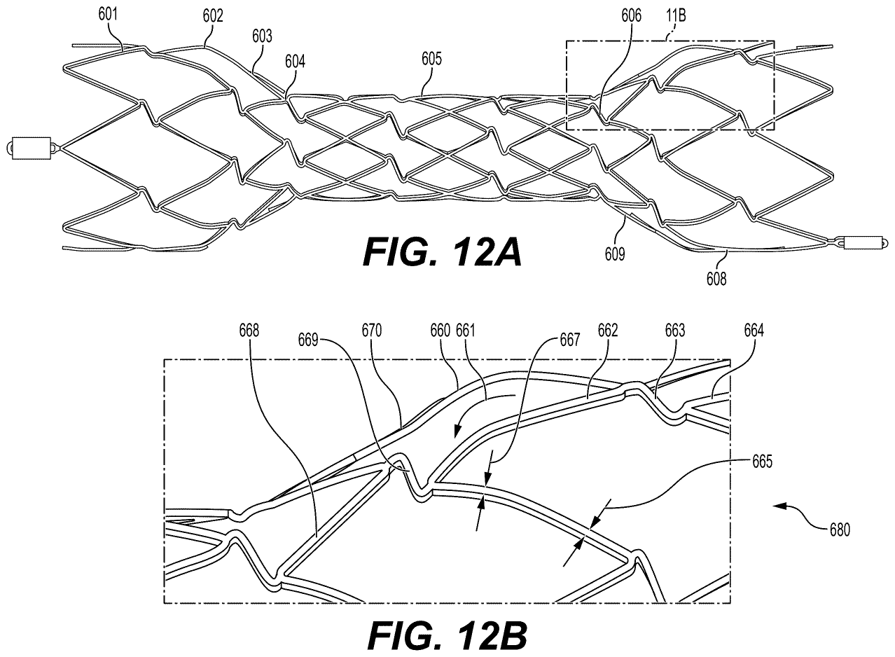

FIG. 12A depicts a side view of an exemplary intracranial stent, according to one or more embodiments of the present invention. The intracranial stent depicted in FIG. 12A may be intracranial stent 600.

FIG. 12B depicts a perspective view of an exemplary connector between struts of the intracranial stent of FIG. 12A, according to one or more embodiments of the present invention. Portion 680 of intracranial stent 600 may include a curved strut 660 at a bend between transition section 609 and end section 608 (or transition section 603 and end section 601). Portion 680 may further include direction 661 indicating the curve of the struts, a tapered strut 662, an end section connector 663, an end section strut 664, strut width 665 at end section 608, strut width 667 at transition section 609, transition section strut 668, and transition section connector 669. In this example, the curvature between transition section 609 and end section 608 may be provided by forming a ring of struts (e.g., a ring of curved struts 660) with a bend out of the plane of intracranial stent 600. The struts (e.g., curved struts 660) may exert a low radial force such that the struts do not apply excessive pressure to the vessel wall in when an eccentric stenosis is present. The strut width (e.g., strut width 667 and/or strut width 665) may be tapered to ensure a smooth increase in radial force from transition section 609 to an anchor ring at the end of end section 608.

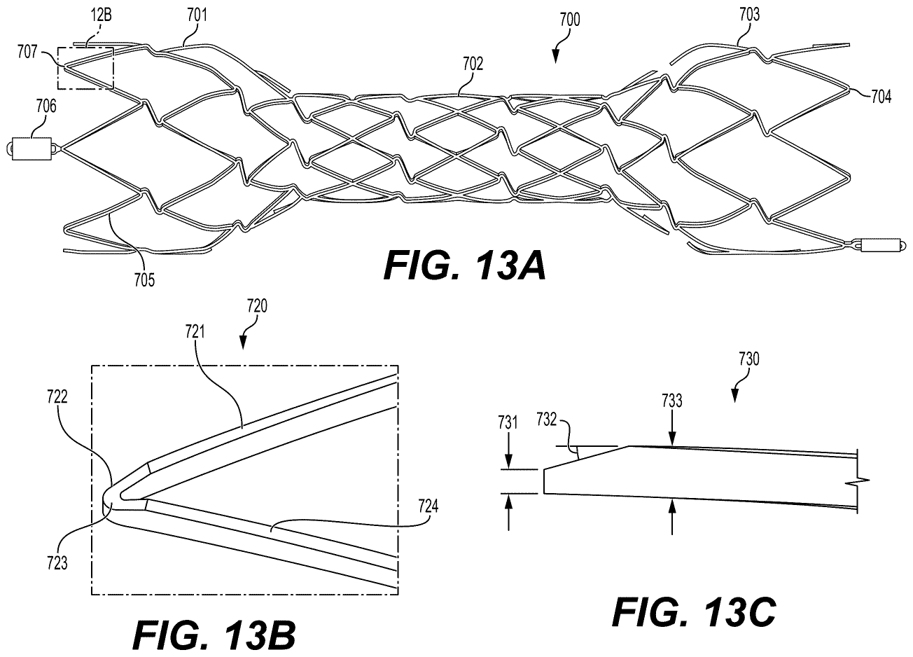

FIG. 13A depicts a side view of an exemplary intracranial stent, according to one or more embodiments of the present invention. Intracranial stent 700 may be similar to any of the previous intracranial stents described herein. Intracranial stent 700 may include a distal end section 701, a midsection 702, a proximal end section 703, a proximal facing crown 704, a distal end section strut 705, a distal radiopaque marker 706, and a distal facing crown 707.

FIG. 13B depicts a perspective view of an exemplary crown and pair of struts of the intracranial stent of FIG. 13A, according to one or more embodiments of the present invention. Portion 720 may include distal facing crown 707, a distal end section strut 721, a chamfered edge 723, and a distal end section strut 724. FIG. 13C depicts a side view of the crown and struts of FIG. 13B, according to one or more embodiments of the present invention. Portion 730 depicts wall thickness 731 at the most distal point of distal facing crown 707 (e.g., at chamfered edge 723), chamfer angle 732 of distal facing crown 707, and stent wall thickness 733. Chamfered edge 723 may reduce the risk of distal facing crown 707 from contacting a PTFE liner of a microcatheter when intracranial stent 700 is pushed from an insertion tool into the microcatheter. Thus, distal facing crown 707 is chamfered to allow distal facing crown 707 to freely advance through a microcatheter.

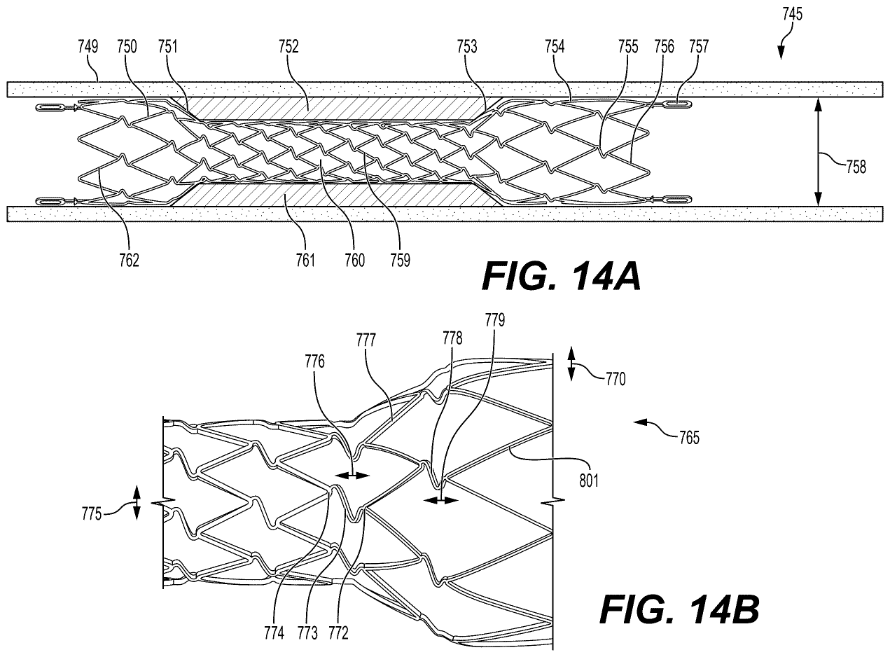

FIGS. 14A and 14B depict side views of an intracranial stent, according to one or more embodiments of the present invention. Intracranial stent 765 may be similar to any of the previous intracranial stents described herein. Arrow 770 may indicate the change in the end section diameter or position of partial intracranial stent 765 due to vessel pulsation. That is, intracranial stent 765 may be configured to expand and constrict (e.g., contract) with the vessel. Intracranial stent 765 may include an end section strut 771, a transition section crown 772, a connector 773 between a transition section and a midsection of partial intracranial stent 765, a midsection crown 774, a transition section strut 777, and a connector 778 between the transition section and the end section of partial intracranial stent 765. Arrow 775 may indicate the change in diameter or position of the midsection due to vessel pulsation. Alternatively, the midsection (e.g., the one or more rings of struts 147 at the central portion 120) may not move during vessel pulsation. Arrow 776 may illustrate how transition section crown 772 and midsection crown 774 can move relative to each other in an axial direction to dampen pulsatile forces from the transition section to the midsection. Arrow 779 may illustrate how the crowns that connector 778 connects may move relative to each other to dampen the transfer of pulsatile forces from the distal end section to the transition section. The dampening of pulsatile forces may be particularly advantageous in at least some embodiments of submaximal stents where the midsection may undergo less pulsatile loading than the end sections. For example, improving the dampening effect of the connectors may improve the fatigue life of the submaximal stent once implanted and reduce the risk of fracture.

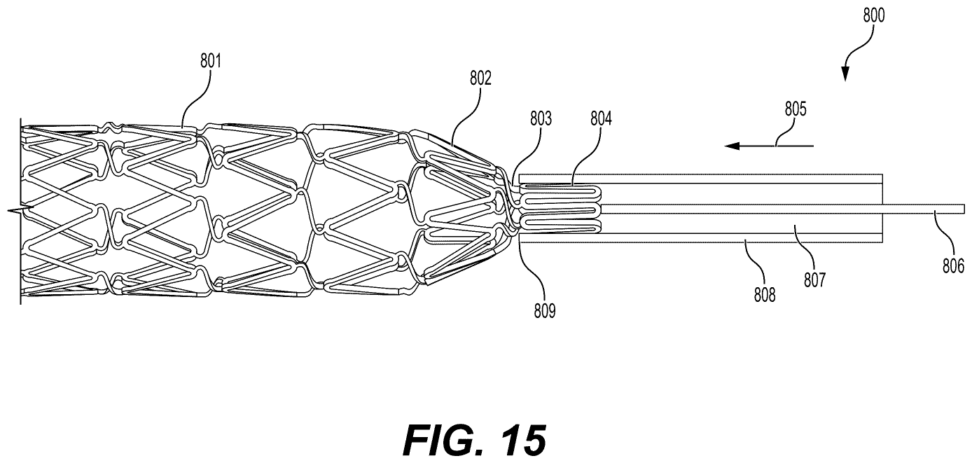

FIG. 15 depicts a perspective view of a portion of an intracranial stent being re-sheathed by a microcatheter, according to one or more embodiments of the present invention. Delivery system 800 illustrates a portion of an intracranial stent 801, a delivery shaft 806 connected to the leading end (e.g., proximal end) of intracranial stent 801, and a microcatheter 808. Intracranial stent 801 may be similar to any of the previous intracranial stents described herein. The rings of intracranial stent 801 may transition from fully expanded to partially collapsed to fully collapsed as intracranial stent 801 is fed into microcatheter 808, with arrow 805 illustrating the direction of movement of microcatheter 808. For example, delivery system 800 illustrates partially collapsed rings 802, connectors 803, and collapsed rings 804. Microcatheter 808 may include a lumen 807 and a distal tip 809.

Delivery system 800 may illustrate a re-sheathing process or a delivery process for intracranial stent 801. Microcatheter 808 may be advanced (e.g., in a distal direction) over intracranial stent 801, causing each ring of intracranial stent 801 to collapse into a loaded configuration. Peak re-sheathing forces may occur when wrapping down each ring of connectors, followed by re-sheathing forces when wrapping down the ring of struts due to contact between these portions of intracranial stent with a distal end face of microcatheter 808.

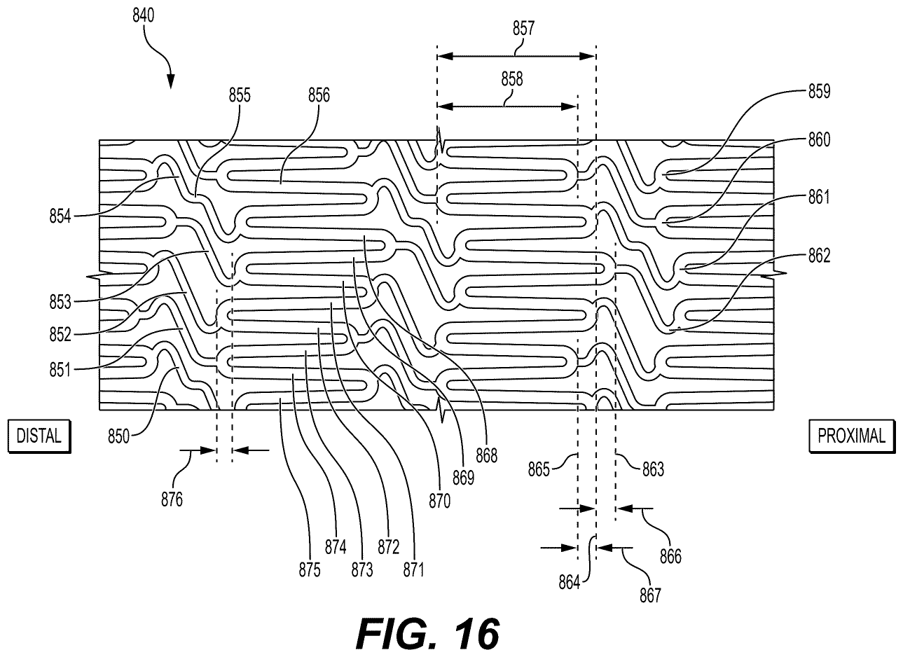

FIG. 16 depicts a two-dimensional projection that represents a portion of an intracranial stent in the loaded configuration, according to one or more embodiments of the present invention. Intracranial stent 840 may be similar to any of the previous intracranial stents described herein. Intracranial stent 840 may include a midsection ring of connectors containing a repeating pattern of four connectors. In an example, the pattern may repeat after two connectors, after five connectors, after six connectors, etc. Intracranial stent 840 includes a connector 850, a connector 851, a connector 852, a connector 853, and a connector 854. Because the pattern of intracranial stent 840 repeats after four connectors, connector 850 and connector 854 may be the same. Connector 850 and connector 854 may include a curved portion, a straight portion, an elbow 855, a second straight portion, and a second curved portion. Connector 851 may include a straight portion, a curved portion, a second straight portion and a second curved portion. Connector 852 and connector 853 may include a first and second curved portion and a straight portion. The length and/or angle of the portions of connector 852 may be different from connector 853.

Intracranial stent 840 may include a strut 856, a strut 868, a strut 869, a strut 870, a strut 871, a strut 872, a strut 873, a strut 874, and a strut 875. The rings of struts of intracranial stent 840 may follow a pattern. For example, the different struts of intracranial stent 840 may repeat after a pattern of eight struts, corresponding with the pattern of four connectors. The struts may be at different positions and have different lengths. For example, the struts may have a long length 857 or a short length 858. Intracranial stent 840 may include a distal crown 859, a distal crown 860, a distal crown 861, a distal crown 862, a staggered crown 863, a staggered crown 864, and a staggered crown 865. Distance 866 may be the distance between the position of staggered crown 863 and staggered crown 864. Distance 867 may be the distance between the position of staggered crown 864 and staggered crown 865. Distance 876 may similarly illustrate the distance between staggered crowns. Staggered crown 863, staggered crown 864, and staggered crown 865 may be part of a first ring of struts and distal crown 859, distal crown 860, distal crown 861, and distal crown 862 may be part of a second ring of struts. Connector 892 may connect staggered crown 864 to distal crown 861. Connector 892 may include a first curved portion 893, a first straight portion 894, an elbow portion 895 at a non-zero angle to the first straight portion 894, a second straight portion 896 that may be parallel to the first straight portion 894, and a second curved portion 897. Connector 898 may connect staggered crown 863 to distal crown 862. Connector 898 may be similar to connector 606 and include a first curved portion, a straight portion, and a second curved portion. Thus, connector 892 may be a different shape than connector 898.

The struts and connectors may be staggered as depicted in FIG. 16 so that when re-sheathing intracranial stent 840, the peak force to wrap down each strut (e.g., struts 856, 868, 869, 870, 871, 872, 873, 874, 875) does not occur at the same time, which may reduce the total peak force. Similarly, the connectors are staggered to potentially reduce the total peak force required to collapse the connectors of intracranial stent 840 and pull them into a microcatheter (e.g., the configuration shown in FIG. 15). While two lengths of struts (e.g., long length 857 and short length 858) may be used for partial intracranial stent 840, three separate proximal crown positions may be created, as is illustrated by distance 866 and distance 867. Using two lengths of struts may improve the deployment stability and radial force distribution around the rings of intracranial stent 840. The connectors may be designed to have two general positions for re-sheathing. Intracranial stent 840 may be a 16 strut ring stent that contains a pattern of eight struts and four connectors repeated twice.

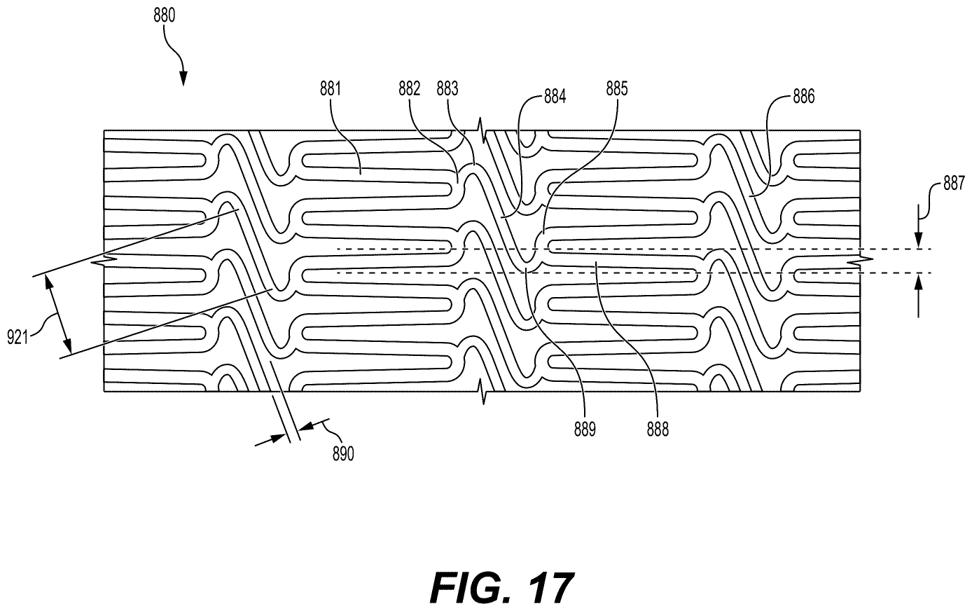

FIG. 17 depicts a two-dimensional project of a portion of an intracranial stent in the loaded configuration, according to one or more embodiments of the present invention. Intracranial stent 880 may be similar to any of the previous intracranial stents described herein. Intracranial stent 880 may include a midsection ring of struts 881, a proximal facing crown 882 between a pair of struts, an upper curved section 883 of a connector, a straight section 884 of a connector, a lower curved section 889 of a connector, a distal facing crown 885 between a pair of struts, a ring of connectors 886 between rings of struts, and a midsection strut 888. Distance 887 may illustrate the distance between the centerline of distal facing crown 885 and the lower curved section 889 of the connector connected to distal facing crown 885. Distance 890 may illustrate the parallel gap between connectors in a collapsed configuration. Distance 891 may illustrate the length of a straight section of a connector (e.g., straight section 884). Distance 891 may be maximized for stent flexibility in collapsed and expanded configurations. As depicted in FIG. 17, the rings of struts and connectors may be aligned with one another instead of offset from one another (e.g., offset as shown in the configuration in FIG. 16). Connectors may be offset by distance 887 from respective crown centerlines (e.g., the centerline of distal facing crown 885) which may determine the maximum distance 891 of the straight sections (e.g., straight section 884) of connectors between crowns. Distance 887 may be in the range of 0.03 mm to 0.20 mm while distance 891 may be in the range of 0.15 mm to 0.50 mm. This may increase the effective beam length of the connector and improve stent flexibility. The gap between connectors (e.g., distance 890) may be sized to allow bending of partial intracranial stent 880 in the collapsed configuration in the microcatheter. Distance 890 may be in the range of 0.01 mm to 0.10 mm.

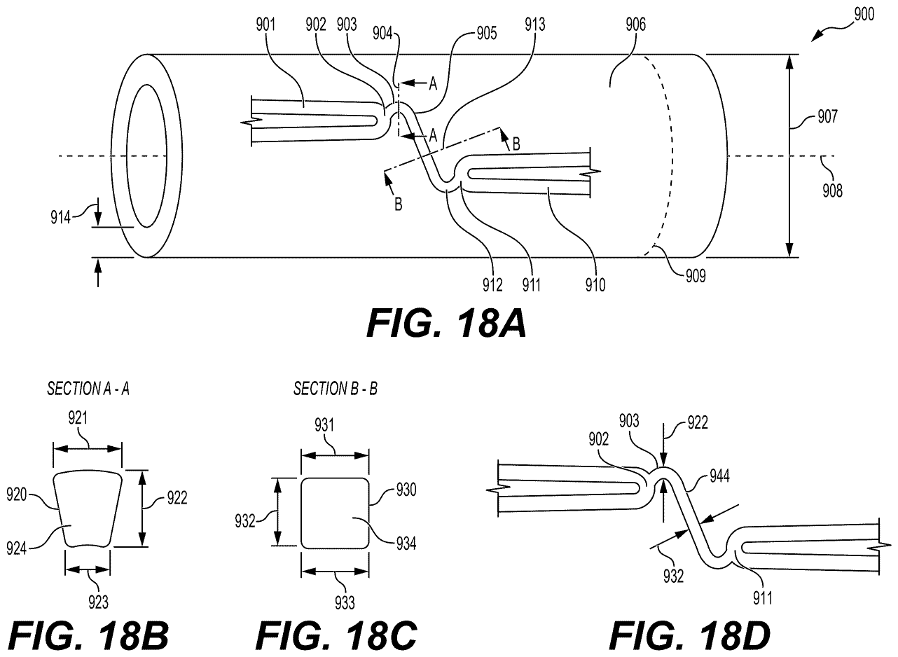

FIGS. 18A and 18D depict a schematic view of a connector, crowns, and struts cut from a raw material tubing, according to one or more embodiments of the present invention. Tubing 900 may be used to form an intracranial stent. Diameter 907 may be the diameter of tubing 900, centerline 908 may be the centerline of tubing 900, dashed line 909 may illustrate the radial direction of tubing 900, and thickness 914 may be the thickness of tubing 900. A strut 901, a crown 902, a connector 905, a strut 910, and a crown 911. Connector 905 may include a curved section 903, a straight section 906, and a curved section 912.

FIG. 18B depicts a cross-section view of Section A-A of FIG. 18A, according to one or more embodiments of the present invention. Cross section 924 of curved section 903 may have opposite sides that are not parallel, and may have curved sides. Outer width 921 of cross section 924 may be larger than an inner width 923 of cross section 924. Inner width 923 may be less than outer width 921 (e.g., cross section 924 is pie-shaped) due to laser cutting of the tube. As will be further described below, since Section A-A is parallel to the radial direction 909, the cross section 924 is pie-shaped instead of rectangular. Wall thickness 922 of cross section 924 may remain substantially the same throughout strut 901, crown 902, connector 905, crown 911, and strut 910.

FIG. 18C depicts a cross-section view of Section B-B of FIG. 18A, according to one or more embodiments of the present invention. Cross section 934 of straight section 906 may be substantially rectangular. For example, adjacent sides of cross section 934 may be perpendicular to one another while opposite sides of cross section 934 may be parallel to one another. Cross section 934 may include an outer width 931, a wall thickness 932, and an inner width 933. Inner width 933 may be approximately equal to outer width 931 when laser cutting raw material tubing 900 since cross section 934 is roughly parallel to centerline 908.

Because the thickness 914 of raw material tubing 900 is small, the cross sectional shape of the connector 905 varies depending on the orientation of the cross section, as illustrated with cross section 924 and cross section 934. If the laser cut made is generally aligned with the radial direction 909, the cross section of the cut may be pie-shaped similar to cross section 924. If the laser cut made is generally aligned with centerline 908, the cross section of the cut may be rectangular similar to cross section 934. The difference in cross sections may be caused by the direction the laser must cut into raw material tubing 900 with respect to the direction the laser must move to make the cut. If a stent is initially designed with a uniform width along the full length of the connector, the curved parts (e.g., curved section 903 and curved section 912) may have a narrower cross section. To compensate for the narrowing cross section, the connector width in the curved parts in the radial direction may be increased to reduce the risk of fracture. That is, the width may be increased to increase durability of the connector. In particular, width 921 may be larger than width 931 and/or width 923 may be substantially equal to width 931.

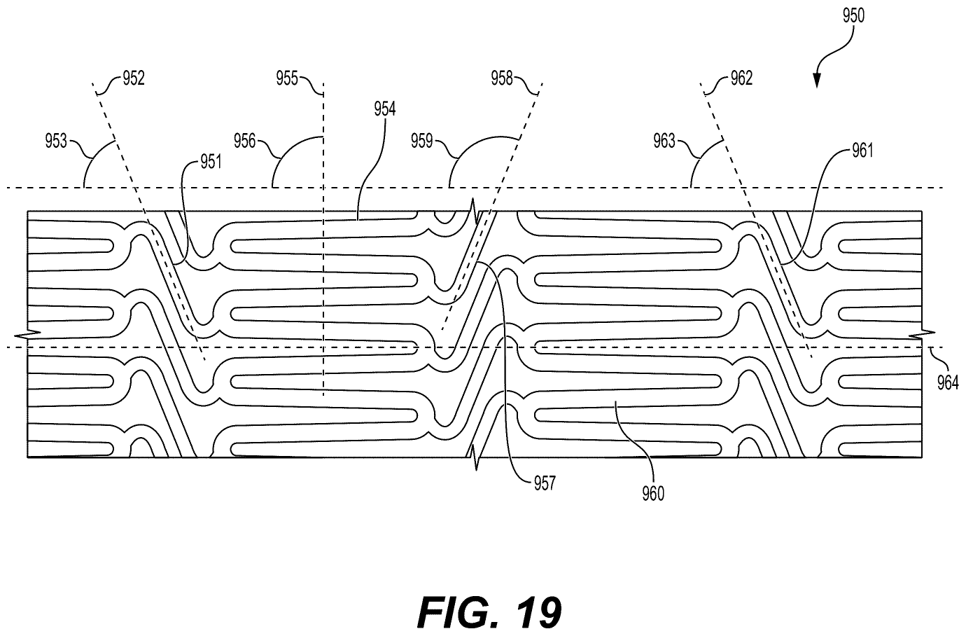

FIG. 19 depicts a two-dimensional projection of a portion of an intracranial stent 950 in a collapsed configuration, according to one or more embodiments of the present invention. Intracranial stent 950 may be similar to any of the previous intracranial stents described herein. Intracranial stent 950 may include a connector 951, a centerline 952 of the straight portion of connector 951, an angle 953 between centerline 952 and a centerline 964 (e.g., axis) of the length of partial intracranial stent 950, a ring of struts 954, a centerline 955 of struts 954 approximately perpendicular to centerline 964, an angle 956 between centerline 955 and centerline 964, a connector 957, a centerline 958 of connector 957, an angle 959 between centerline 958 and centerline 964, a ring of struts 960 adjacent to struts 954 (e.g., adjacent such that connector 957 connects struts 960 and struts 954), a connector 961, a centerline 962 of connector 961, and an angle 963 between centerline 962 and centerline 964. As depicted in FIG. 19, the direction of connectors may alternate between each ring of struts to minimize a twisting moment (e.g., torque) applied to partial intracranial stent 950 by a microcatheter during deployment or re-sheathing. For example, centerline 952 may extend parallel to centerline 962 while centerline 952 and centerline 958 (e.g., centerlines of adjacent rings of connectors) may be mirrored about centerline 955. Similarly, angle 953 may be the same as angle 963. Angle 953 and angle 959 may be supplementary angles (e.g., angle 953 and angle 959 add up to 180 degrees). In general, rings of connectors may alternate in direction such that adjacent rings of connectors are supplementary angles.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An intracranial stent, comprising:
one or more rings of struts at a proximal end portion of the intracranial stent;
one or more rings of struts at a distal end portion of the intracranial stent;
one or more rings of struts at a central portion of the intracranial stent having a diameter that is smaller than a diameter of the one or more rings of struts at the proximal end portion and that is smaller than a diameter of the one or more rings of struts at the distal end portion;
one or more rings of struts at a proximal transition portion between the proximal end portion and the central portion;
one or more rings of struts at a distal transition portion between the distal end portion and the central portion; and
a first ring of connectors connecting a first ring of the one or more rings of struts at the proximal end portion with a second ring of the one or more rings of struts at the proximal transition portion, wherein each connector of the first ring of connectors includes a first curved section, a straight section, and a second curved section.

2. The intracranial stent of claim 1, the one or more rings of struts at the central portion including a first ring and a second ring, wherein the first ring is directly connected to the second ring.

3. The intracranial stent of claim 1, further comprising:
a radiopaque marker including an eyelet portion and a marker engagement strut, wherein the one or more rings of struts at the proximal end portion include a proximal end ring, wherein the marker engagement strut is connected to a crown of the proximal end ring.

4. The intracranial stent of claim 1, wherein the one or more rings of struts at the distal end portion include a distal end ring, and wherein one or more crowns of the distal end ring are chamfered.

5. The intracranial stent of claim 1, wherein the one or more rings of struts at the proximal transition portion gradually increase in diameter from the central portion towards the proximal end portion, and wherein the one or more rings of struts at the distal transition portion gradually increase in diameter from the central portion towards the distal end portion.

6. The intracranial stent of claim 1, wherein each connector of a second ring of connectors at the distal end portion has a smaller radius of curvature than each connector of a third ring of connectors at the central portion.

7. The intracranial stent of claim 1, wherein a distance between the struts at the distal end portion, as measured in a longitudinal direction, is greater than a distance between the struts at the central portion.

8. The intracranial stent of claim 1, wherein the distal end portion and the proximal end portion are configured to exert a lower radial force than the central portion.

9. The intracranial stent of claim 1, wherein a radial force profile across a length of the intracranial stent is asymmetrical.

10. The intracranial stent of claim 1, wherein a radial force profile across a length of the intracranial stent is symmetrical.

11. The intracranial stent of claim 1, wherein the one or more rings of struts at one of the end portions is configured to move towards and away from the one or more rings of struts at one of the transition portions due to radial expansion and contraction of one of the end portions.

12. The intracranial stent of claim 1, wherein adjacent rings of connectors alternate in direction.

13. The intracranial stent of claim 8, wherein a radial force applied by the central portion is substantially equal along an entire length of the central portion.

14. The intracranial stent of claim 9, wherein the central portion includes a first section and a second section, wherein the first section of the central portion is closer to a distal end of the intracranial stent than the second section of the central portion, and wherein the second section of the central portion exerts a higher radial force than the first section of the central portion.

15. The intracranial stent of claim 3, wherein the radiopaque marker includes an engagement member that protrudes laterally outward from the marker engagement strut.

16. The intracranial stent of claim 1, further comprising:
a distal end radiopaque marker, wherein a radial thickness of the distal end radiopaque marker is greater than a radial thickness of the one or more rings of struts at the distal end portion.

17. The intracranial stent of claim 15, wherein a radial thickness of the one or more rings of struts at the central portion is greater than the radial thickness of the one or more rings of struts at the distal end portion.

18. The intracranial stent of claim 16, wherein a radial thickness of the one or more rings of struts at the distal transition portion gradually increases from the radial thickness of the one or more rings of struts at the distal end portion to the radial thickness of the one or more rings of struts at the central portion.

19. The intracranial stent of claim 1, wherein the one or more rings of struts at the distal end portion of the intracranial stent have a first wall thickness, wherein the one or more rings of struts between the distal end portion and the distal transition portion have a second wall thickness, and wherein the first wall thickness is greater than the second wall thickness.

20. An intracranial stent, comprising:
a proximal end portion;
a distal end portion;
a central portion having a diameter that is smaller than a diameter of the proximal end portion and that is smaller than a diameter of the distal end portion;
a plurality of struts, wherein a first group of struts is connected to define a first ring of struts, and wherein a second group of struts is connected to define a second ring of struts; and
a plurality of connectors, wherein each connector of the plurality of connectors includes a longitudinal axis, wherein a first group of the plurality of connectors connects the first ring of struts to the second ring of struts.

21. The intracranial stent of claim 20, wherein each connector of the first group of the plurality of connectors is configured to twist about the longitudinal axis, and further wherein the connector of the first group of the plurality of connectors is located between the proximal end portion and the central portion.

22. The intracranial stent of claim 20, wherein a distance between the first ring of struts and the second ring of struts is configured to increase and/or decrease as the first group of the plurality of connectors lengthen and shorten, respectively.

23. The intracranial stent of claim 20, wherein a third group of the plurality of struts are connected to define a third ring of struts, wherein the third ring of struts is directly connected to the first ring of struts.

24. A method of treating an intracranial atherosclerotic stenosis in an intracranial blood vessel, the method comprising:
deploying an intracranial stent in the intracranial blood vessel, the intracranial stent including:
a proximal end portion;
a distal end portion;
a central portion having a diameter that is smaller than a diameter of the proximal end portion and that is smaller than a diameter of the distal end portion; and
a first ring of connectors connecting a first ring of struts at the central portion with a second ring of struts at the central portion, wherein each connector of the first ring of connectors includes a first curved section, a straight section, and a second curved section.

25. The method of claim 24, wherein a cross-sectional shape of each connector of the first ring of connectors includes a substantially pie-shaped section connected to a substantially rectangular section.
